(19) United States
(12) Patent Application Publication
(10) Pub. No.: US 2024/0029874 A1
(43) Pub. Date: Jan. 25, 2024

(54) WORK SUPPORT APPARATUS, WORK SUPPORT METHOD, AND WORK SUPPORT PROGRAM

(71) Applicant: FUJIFILM Corporation, Tokyo (JP)

(72) Inventors: Takuya YUZAWA, Tokyo (JP); Eiichi IMAMICHI, Tokyo (JP)

(73) Assignee: FUJIFILM Corporation, Tokyo (JP)

(21) Appl. No.: 18/481,260

(22) Filed: Oct. 5, 2023

Related U.S. Application Data
(63) Continuation of application No. PCT/JP2022/016718, filed on Mar. 31, 2022.

(30) Foreign Application Priority Data
Apr. 14, 2021 (JP) 2021-068675
Dec. 22, 2021 (JP) 2021-208526

(51) Int. Cl. G16H 40/20; G16H 30/20; G16H 40/63; G16H 15/00

(52) U.S. Cl. CPC G16H 40/20; G16H 30/20; G16H 40/63; G16H 15/00

(57) ABSTRACT

A work support apparatus acquires a medical image, and performs control to display, in an identifiable manner, a status for a region that is likely to be a target of at least one work of interpretation work or creation work of a medical document by a user in the medical image among a plurality of statuses related to the work by the user.

### CROSS-REFERENCE TO RELATED APPLICATIONS

This application is a continuation of International Application No. PCT/JP2022/016718, filed on Mar. 31, 2022, which claims priority from Japanese Patent Application No. 2021-068675, filed on Apr. 14, 2021 and Japanese Patent Application No. 2021-208526, filed on Dec. 22, 2021. The entire disclosure of each of the above applications is incorporated herein by reference.

### BACKGROUND

### 1. Technical Field

The present disclosure relates to a work support apparatus, a work support method, and a work support program.

### 2. Description of the Related Art

In the related art, there have been proposed technologies for improving the efficiency of creation of a medical document such as an interpretation report by a doctor. JP1995-323024A (JP-H7-323024A) discloses a technology of obtaining a part indicated by coordinates on a medical image designated by a doctor from the designated coordinates and data obtained by dividing the medical image into regions for each part, and outputting the part with an abnormality and a name of a disease.

In addition, JP1995-031591A (JP-H7-031591A) discloses a technology of detecting a type and a position of an abnormality included in a medical image and generating an interpretation report including the detected type and position of the abnormality based on fixed phrases.

### SUMMARY

In a case where there are a plurality of regions in a medical image that are likely to be a target of work such as interpretation work and creation work of an interpretation report by a user such as a doctor, it is preferable for the user to be able to ascertain a work status such as how far the work has progressed for each region. However, with the technologies disclosed in JP1995-323024A (JP-H7-323024A) and JP1995-031591A (JP-H7-031591A), although it is possible to detect a region such as an abnormal shadow that is likely to be a work target by a user from a medical image, it is not possible to ascertain the user's work status for each region.

The present disclosure has been made in view of the above circumstances, and an object of the present disclosure is to provide a work support apparatus, a work support method, and a work support program capable of ascertaining a user's work status for each region included in a medical image that is likely to be a work target by the user.

According to an aspect of the present disclosure, there is provided a work support apparatus comprising at least one processor, in which the processor is configured to: acquire a medical image; and perform control to display, in an identifiable manner, a status for a region that is likely to be a target of at least one work of interpretation work or creation work of a medical document by a user in the medical image among a plurality of statuses related to the work by the user.

In addition, in the work support apparatus according to the aspect of the present disclosure, the plurality of statuses may include at least one of a status in which the work is required or a status in which the work is not required.

In addition, in the work support apparatus according to the aspect of the present disclosure, the plurality of statuses may include two or more of a status in which the user has not confirmed the region, a status in which the user has designated the region as a target of the work and the work is incomplete, a status in which the user has designated that the region is excluded from the target of the work, and a status that the work for the region is completed.

In addition, in the work support apparatus according to the aspect of the present disclosure, the processor may be configured to, in a case where the status is the status in which the user has designated the region as the target of the work and the work is incomplete, perform control to display the status in an identifiable manner by adding a predetermined mark to the region.

In addition, in the work support apparatus according to the aspect of the present disclosure, the processor may be configured to perform control to display the status in which the user has not confirmed the region and the status in which the user has designated the region as the target of the work and the work is incomplete among the plurality of statuses to be different from other statuses.

In addition, in the work support apparatus according to the aspect of the present disclosure, the processor may be configured to further perform control to display information regarding each of a plurality of the regions.

In addition, in the work support apparatus according to the aspect of the present disclosure, the processor may be configured to perform control to display a list of the information regarding each of the plurality of regions.

In addition, in the work support apparatus according to the aspect of the present disclosure, the processor may be configured to perform control to display the list of the information regarding each of the plurality of regions for each of the statuses.

In addition, in the work support apparatus according to the aspect of the present disclosure, the region may be a region including an abnormal shadow.

In addition, in the work support apparatus according to the aspect of the present disclosure, the region may be extracted from the medical image by an extraction process via a computer.

In addition, in the work support apparatus according to the aspect of the present disclosure, the region may be a region designated by the user.

In addition, in the work support apparatus according to the aspect of the present disclosure, the processor may be configured to further perform control to display a presence or absence of a change from the same region detected in a past examination for the region in an identifiable manner.

In addition, in the work support apparatus according to the aspect of the present disclosure, the processor may be configured to further perform control to display whether or not the same region has been detected in a past examination for the region in an identifiable manner.

In addition, in the work support apparatus according to the aspect of the present disclosure, the processor may be configured to further perform control to display a presence or absence of a change from the same region reported in a past examination for the region in an identifiable manner.

In addition, in the work support apparatus according to the aspect of the present disclosure, the processor may be configured to further perform control to display whether or not the same region has been reported in a past examination for the region in an identifiable manner.

In addition, according to another aspect of the present disclosure, there is provided a work support method executed by a processor provided in a work support apparatus, the method comprising: acquiring a medical image; and performing control to display, in an identifiable manner, a status for a region that is likely to be a target of at least one work of interpretation work or creation work of a medical document by a user in the medical image among a plurality of statuses related to the work by the user.

In addition, according to another aspect of the present disclosure, there is provided a work support program for causing a processor provided in a work support apparatus to execute: acquiring a medical image; and performing control to display, in an identifiable manner, a status for a region that is likely to be a target of at least one work of interpretation work or creation work of a medical document by a user in the medical image among a plurality of statuses related to the work by the user.

According to the aspects of the present disclosure, it is possible to ascertain a user's work status for each region included in a medical image that is likely to be a work target by the user.

### BRIEF DESCRIPTION OF THE DRAWINGS

FIG. 1 is a block diagram showing a schematic configuration of a medical information system.

FIG. 2 is a block diagram showing an example of a hardware configuration of a work support apparatus.

FIG. 3 is a block diagram showing an example of a functional configuration of the work support apparatus.

FIG. 4 is a diagram showing an example of a comment-on-findings display screen.

FIG. 5 is a diagram showing an example of a comment-on-findings display screen.

FIG. 6 is a diagram showing an example of a status display screen.

FIG. 7 is a flowchart showing an example of a work support process.

FIG. 8 is a diagram showing an example of a list display screen.

FIG. 9 is a diagram showing an example of a status display screen according to a modification example.

FIG. 10 is a diagram showing an example of a status display screen according to a modification example.

FIG. 11 is a diagram showing an example of a status display screen according to a modification example.

FIG. 12 is a diagram showing an example of a display screen in a pop-up format.

FIG. 13 is a diagram showing an example of a display screen in a tab format.

FIG. 14 is a diagram showing an example of a status display screen according to a modification example.

FIG. 15 is a diagram showing an example of a status display screen according to a modification example.

### DETAILED DESCRIPTION

Hereinafter, form examples for implementing a technology of the present disclosure will be described in detail with reference to the drawings.

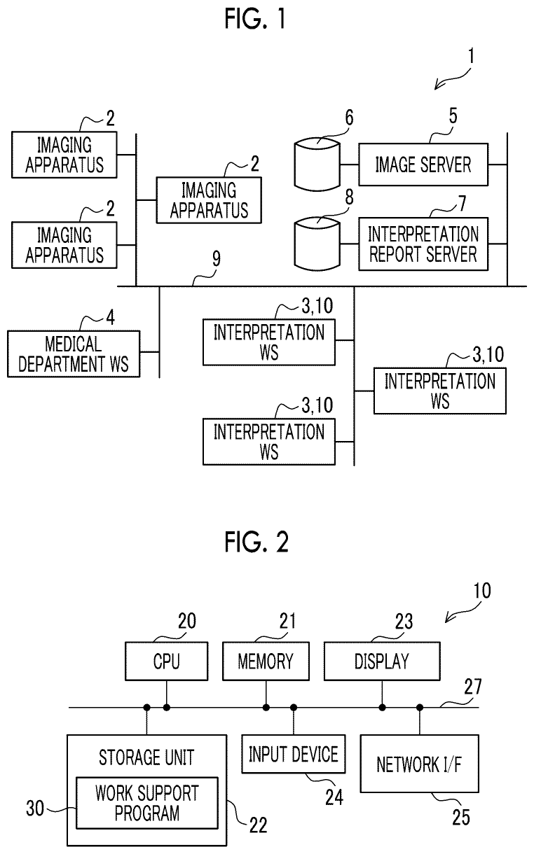

First, a configuration of a medical information system 1 to which a work support apparatus according to the disclosed technology is applied will be described with reference to FIG. 1. The medical information system 1 is a system for performing imaging of a diagnosis target part of a subject and storing of a medical image acquired by the imaging based on an examination order from a doctor in a medical department using a known ordering system. In addition, the medical information system 1 is a system for performing interpretation of a medical image and creation of an interpretation report by a radiologist, and viewing the interpretation report and detailed observation of the medical image to be interpreted by a doctor of a medical department that is a request source.

As shown in FIG. 1, the medical information system 1 according to the present embodiment includes a plurality of imaging apparatuses 2, a plurality of interpretation workstations (WS) 3 that are interpretation terminals, a medical department WS 4, an image server 5, an image database (DB) 6, an interpretation report server 7, and an interpretation report DB 8. The imaging apparatus 2, the interpretation WS 3, the medical department WS 4, the image server 5, and the interpretation report server 7 are connected to each other via a wired or wireless network 9 in a communicable state. In addition, the image DB 6 is connected to the image server 5, and the interpretation report DB 8 is connected to the interpretation report server 7.

The imaging apparatus 2 is an apparatus that generates a medical image showing a diagnosis target part of a subject by imaging the diagnosis target part. The imaging apparatus 2 may be, for example, a simple X-ray imaging apparatus, an endoscope apparatus, a computed tomography (CT) apparatus, a magnetic resonance imaging (MRI) apparatus, a positron emission tomography (PET) apparatus, and the like. A medical image generated by the imaging apparatus 2 is transmitted to the image server 5 and is saved therein.

The medical department WS 4 is a computer used by a doctor in the medical department for detailed observation of a medical image, viewing of an interpretation report, creation of an electronic medical record, and the like. In the medical department WS 4, each process such as creating an electronic medical record of a patient, requesting the image server 5 to view an image, and displaying a medical image received from the image server 5 is performed by executing a software program for each process. In addition, in the medical department WS 4, each process such as automatically detecting or highlighting suspected disease regions in the medical image, requesting to view an interpretation report from the interpretation report server 7, and displaying the interpretation report received from the interpretation report server 7 is performed by executing a software program for each process.

The image server 5 incorporates a software program that provides a function of a database management system (DBMS) to a general-purpose computer. In a case where the image server 5 receives a request to register a medical image from the imaging apparatus 2, the image server 5 prepares the medical image in a format for a database and registers the medical image in the image DB 6.

Image data representing the medical image acquired by the imaging apparatus 2 and accessory information attached to the image data are registered in the image DB 6. The accessory information includes information such as an image identification (ID) for identifying individual medical images, a patient ID for identifying a patient who is a subject, an examination ID for identifying examination content, and a unique identification (UID) assigned to each medical image, for example. In addition, the accessory information includes information such as an examination date when a medical image was generated, an examination time, the type of imaging apparatus used in the examination for acquiring the medical image, patient information (for example, a name, an age, and a gender of the patient), an examination part (that is, an imaging part), and imaging information (for example, an imaging protocol, an imaging sequence, an imaging method, imaging conditions, and whether or not a contrast medium is used), and a series number or collection number when a plurality of medical images are acquired in one examination. In addition, in a case where a viewing request from the interpretation WS 3 is received through the network 9, the image server 5 searches for a medical image registered in the image DB 6 and transmits the searched for medical image to the interpretation WS 3 that is a request source.

The interpretation report server 7 incorporates a software program for providing a function of DBMS to a general-purpose computer. In a case where the interpretation report server 7 receives a request to register an interpretation report from the interpretation WS 3, the interpretation report server 7 prepares the interpretation report in a format for a database and registers the interpretation report in the interpretation report database 8. Further, in a case where the request to search for the interpretation report is received, the interpretation report is searched for from the interpretation report DB 8.

In the interpretation report DB 8, for example, an interpretation report is registered in which information, such as an image ID for identifying a medical image to be interpreted, a radiologist ID for identifying an image diagnostician who performed the interpretation, a lesion name, position information of a lesion, findings, and a degree of certainty of the findings, is recorded.

The network 9 is a wired or wireless local area network that connects various apparatuses in a hospital to each other. In a case where the interpretation WS 3 is installed in another hospital or clinic, the network 9 may be configured to connect local area networks of respective hospitals through the Internet or a dedicated line. In any case, it is preferable that the network 9 has a configuration capable of realizing high-speed transmission of medical images such as an optical network.

The interpretation WS 3 requests the image server 5 to view a medical image, performs various types of image processing on the medical image received from the image server 5, displays the medical image, performs an analysis process on the medical image, highlights the medical image based on an analysis result, and creates an interpretation report based on the analysis result. In addition, the interpretation WS 3 supports creation of an interpretation report, requests the interpretation report server 7 to register and view an interpretation report, displays the interpretation report received from the interpretation report server 7, and the like. The interpretation WS 3 performs each of the above processes by executing a software program for each process. The interpretation WS 3 encompasses a work support apparatus 10, which will be described later, and in the above processes, processes other than those performed by the work support apparatus 10 are performed by a well-known software program, and therefore the detailed description thereof will be omitted here. In addition, processes other than the processes performed by the work support apparatus 10 may not be performed in the interpretation WS 3, and a computer that performs the processes may be separately connected to the network 9, and in response to a processing request from the interpretation WS 3, the requested process may be performed by the computer. Hereinafter, the work support apparatus 10 encompassed in the interpretation WS 3 will be described in detail.

Next, a hardware configuration of the work support apparatus 10 according to the present embodiment will be described with reference to FIG. 2. As shown in FIG. 2, the work support apparatus 10 includes a central processing unit (CPU) 20, a memory 21 as a temporary storage area, and a non-volatile storage unit 22. Further, the work support apparatus 10 includes a display 23 such as a liquid crystal display, an input device 24 such as a keyboard and a mouse, and a network interface (UF) 25 connected to the network 9. The CPU 20, the memory 21, the storage unit 22, the display 23, the input device 24, and the network OF 25 are connected to a bus 27.

The storage unit 22 is realized by a hard disk drive (HDD), a solid state drive (SSD), a flash memory, or the like. A work support program 30 is stored in the storage unit 22 as a storage medium. The CPU 20 reads out the work support program 30 from the storage unit 22, loads the read work support program 30 into the memory 21, and executes the loaded work support program 30.

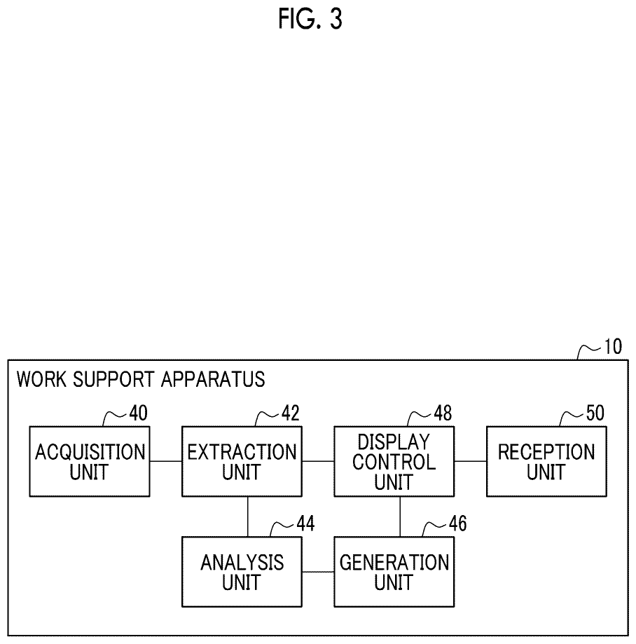

Next, a functional configuration of the work support apparatus 10 according to the present embodiment will be described with reference to FIG. 3. As shown in FIG. 3, the work support apparatus 10 includes an acquisition unit 40, an extraction unit 42, an analysis unit 44, a generation unit 46, a display control unit 48, and a reception unit 50. The CPU 20 executes the work support program 30 to function as the acquisition unit 40, the extraction unit 42, the analysis unit 44, the generation unit 46, the display control unit 48, and the reception unit 50.

The acquisition unit 40 acquires a medical image to be diagnosed (hereinafter referred to as a “diagnosis target image”) from the image server 5 via the network OF 25. In the following, a case where the diagnosis target image is a CT image of the liver will be described as an example.

The extraction unit 42 extracts a region in the diagnosis target image acquired by the acquisition unit 40 that is likely to be the target of the creation work of a medical document by a user such as a doctor. Examples of medical documents include interpretation reports and the like. In the present embodiment, an example in which a region including an abnormal shadow is applied as regions that are likely to be a target of creation work of a medical document will be described, but the present disclosure is not limited thereto. For example, regions of organs such as the lung and the liver may be applied as regions that are likely to be a target of creation work of a medical document, or regions of an anatomical structure such as subsegments divided into S1 to S8 of the liver may be applied.

In the present embodiment, an example in which a region that is likely to be a target of creation work of a medical document is applied to as a region to be extracted by the extraction unit 42 will be described, but the present disclosure is not limited thereto. For example, as the region to be extracted by the extraction unit 42, a region that is likely to be the target of the interpretation work may be applied or a region that is likely to be the target of both the interpretation work and the creation work of the medical document may be applied.

Specifically, the extraction unit 42 extracts a region including an abnormal shadow using a trained model M1 for detecting the abnormal shadow from the diagnosis target image. The abnormal shadow refers to a shadow suspected of having a disease such as a nodule. The trained model M1 is configured by, for example, a convolutional neural network (CNN) that receives a medical image as an input and outputs information regarding an abnormal shadow included in the medical image. The trained model M1 is, for example, a model trained by machine learning using, as training data, a large number of combinations of a medical image including an abnormal shadow and information specifying a region in the medical image in which the abnormal shadow is present.

The extraction unit 42 inputs the diagnosis target image to the trained model Ml. The trained model M1 outputs information specifying a region in which an abnormal shadow included in the input diagnosis target image is present. In addition, the extraction unit 42 may extract a region including an abnormal shadow by a known computer-aided diagnosis (CAD), or may extract a region designated by the user as a region including the abnormal shadow. In addition, a region extraction process by the extraction unit 42 may be executed by an external computer such as the medical department WS 4. In this case, the extraction unit 42 is provided in the external computer.

The analysis unit 44 analyzes each of the abnormal shadows extracted by the extraction unit 42, and derives findings of the abnormal shadows. Specifically, the extraction unit 42 derives the findings of the abnormal shadow using a trained model M2 for deriving the findings of the abnormal shadow. The trained model M2 is configured by, for example, a CNN that receives, for example, a medical image including an abnormal shadow and information specifying a region in the medical image in which the abnormal shadow is present as inputs, and outputs a finding of the abnormal shadow. The trained model M2 is, for example, a model trained by machine learning using, as training data, a large number of combinations of a medical image including an abnormal shadow, information specifying a region in the medical image in which the abnormal shadow is present, and a finding of the abnormal shadow.

The analysis unit 44 inputs, to the trained model M2, information specifying a diagnosis target image and a region in which the abnormal shadow extracted by the extraction unit 42 for the diagnosis target image is present. The trained model M2 outputs findings of the abnormal shadow included in the input diagnosis target image. Examples of the findings of the abnormal shadow include the position, size, presence or absence of calcification, benign or malignant, presence or absence of irregular margin, type of disease, and the like.

The generation unit 46 generates a plurality of comments on findings based on the findings derived by the analysis unit 44. Specifically, for example, the generation unit 46 generates a plurality of comments on findings by inputting the findings to a recurrent neural network trained to generate text from input words. As an example of a plurality of comments on findings, there are a plurality of comments on findings having items of different findings such as “Malignant tumor of o mm is found in the liver S6” and “Tumor of o mm is found in the liver S6”. Note that the plurality of comments on findings may be a plurality of comments on findings having the same meaning but different expressions.

The display control unit 48 performs control to display information indicating a region extracted by the extraction unit 42 on the display 23. In addition, the display control unit 48 performs control to display, in an identifiable manner, a status for the region extracted by the extraction unit 42 among a plurality of statuses related to creation work of a medical document by a user. In the present embodiment, an example will be described in which the following four statuses are applied as a plurality of statuses related to the creation work of a medical document.

A first status is a status in which the user has not confirmed the region extracted by the extraction unit 42. A second status is a status in which the user has designated that the region extracted by the extraction unit 42 is the target of the creation work of the medical document, and the creation work is incomplete. A third status is a status in which the user has designated that the region extracted by the extraction unit 42 is excluded from the creation work of the medical document. A fourth status is a status in which the creation work of the medical document for the region extracted by the extraction unit 42 has been completed. Note that the plurality of statuses may be two or three of these four statuses.

The first status and the second status correspond to statuses in which the creation work of a medical document is required. In addition, the third status and the fourth status correspond to statuses in which the creation work of a medical document is not required. The fourth status is a status in which the creation work of the medical document is no longer necessary because the creation work of the medical document is completed.

The display control unit 48 performs control to display the first status in an identifiable manner in the control of first displaying the information indicating the region extracted by the extraction unit 42 on the display 23. Specifically, for example, the display control unit 48 performs control to display the diagnosis target image on the display 23 in a state where the region extracted by the extraction unit 42 in the diagnosis target image is filled with a preset color. Note that, at the time of this control, in a case where a plurality of abnormal shadows having different disease types are extracted by the extraction unit 42, the display control unit 48 may display the disease types in an identifiable manner by making colors different for each disease type.

In a case where an operation in which a region is designated by the user and the status of the designated region is set as the second status is performed, the display control unit 48 performs control to display the second status in an identifiable manner by adding a predetermined mark to the region. Accordingly, a region which is designated by the user as a target for creating the medical document but in which the creation of the medical document has not been completed is conspicuous, so that it is possible to suppress omission of the creation of the medical document.

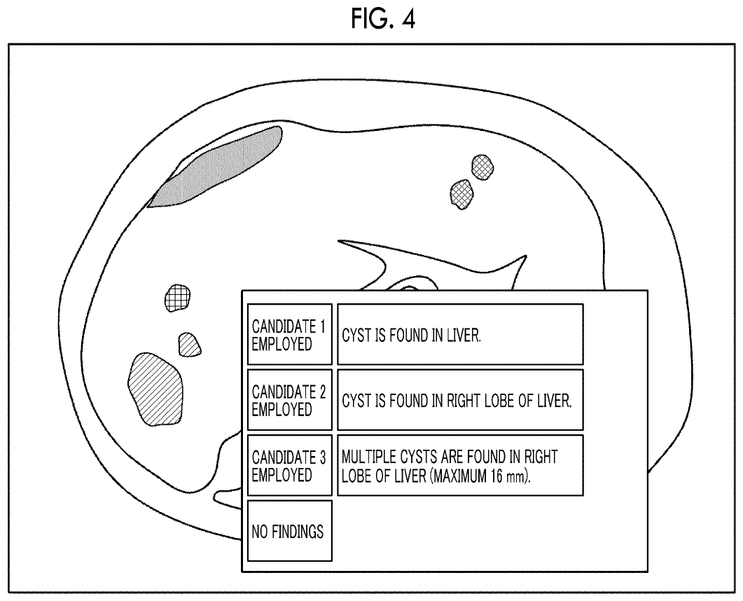

In a case where an operation in which a region is designated by the user and an instruction for displaying the comment on findings is provided is performed, the display control unit 48 performs control to display, on the display 23, a plurality of comments on findings generated by the generation unit 46 for the region. FIG. 4 shows an example of a comment-on-findings display screen displayed on the display 23 by this control. As shown in FIG. 4, on the comment-on-findings display screen, a plurality of comments on findings, a button designated in a case where the user selects each comment on findings, and a button designated in a case where the user determines that there is no finding are displayed. In a case where the user performs an operation of selecting one comment on findings from a plurality of comments on findings on the comment-on-findings display screen, the display control unit 48 performs control to display the fourth status in an identifiable manner by canceling the filling of the region and drawing the outer edge of the region with lines of a predetermined color.

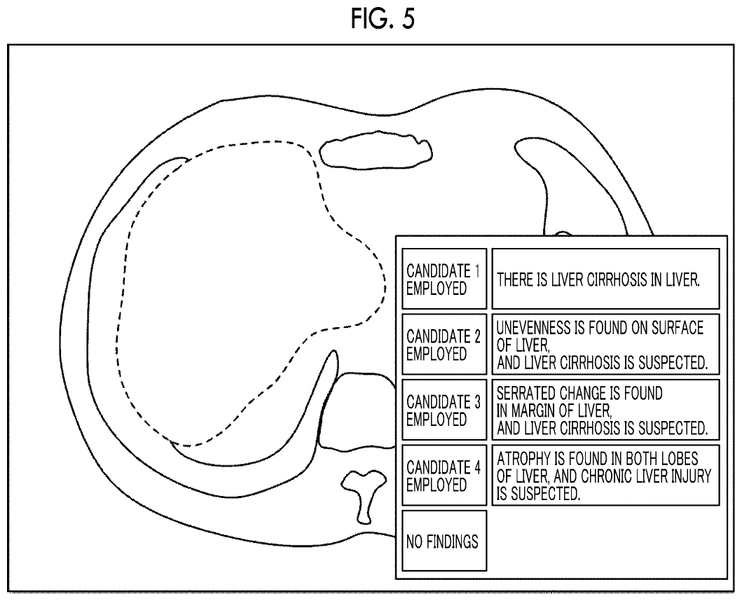

FIG. 5 shows another example of the comment-on-findings display screen. FIG. 4 is an example in a case where an abnormal shadow is detected in the liver, and FIG. 5 is an example in a case where the shape of the liver itself is detected as an abnormal shadow.

In a case where an operation in which a region is designated by the user and the status of the designated region is set as the third status is performed, the display control unit 48 performs control to display the third status in an identifiable manner by graying out the region. An example of this operation includes an operation in which the user designates a no-finding button on the comment-on-findings display screen shown in FIG. 4, for example.

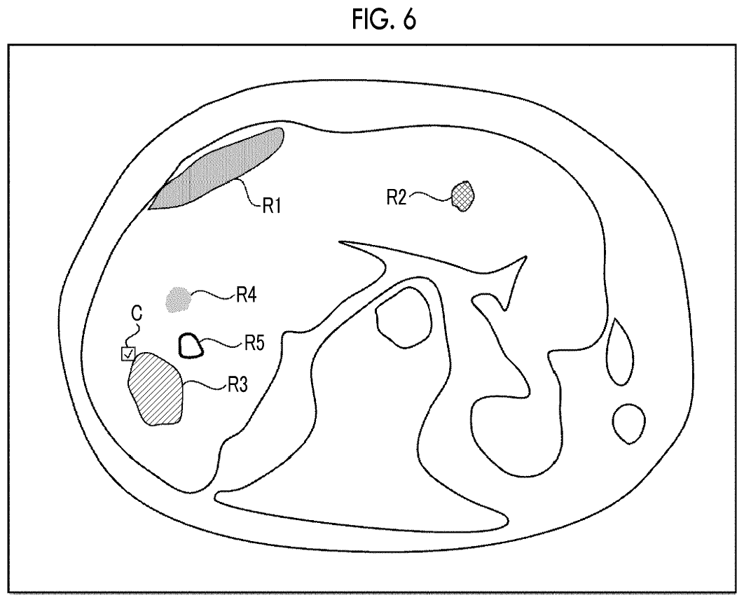

FIG. 6 shows an example of a status display screen displayed on the display 23 under the control of the display control unit 48. FIG. 6 shows an example in which the statuses of a region R1 and a region R2 are the first status, and the status of a region R3 is the second status. A check mark C is added to the region R3 as a predetermined mark. FIG. 6 shows an example in which the status of a region R4 is the third status and a status of the region R5 is the fourth status.

Note that the method of displaying the first status to the fourth status in an identifiable manner is not limited to the above example. For example, the first to fourth statuses may be displayed in an identifiable manner by the line type or thickness of the contour of the region, the transparency or pattern of the filling of the region, the blinking of the region, the animation display of the region, the addition of different marks, and the like. Also, the mark is not limited to the check mark, and may be an arrow or a symbol such as “+”.

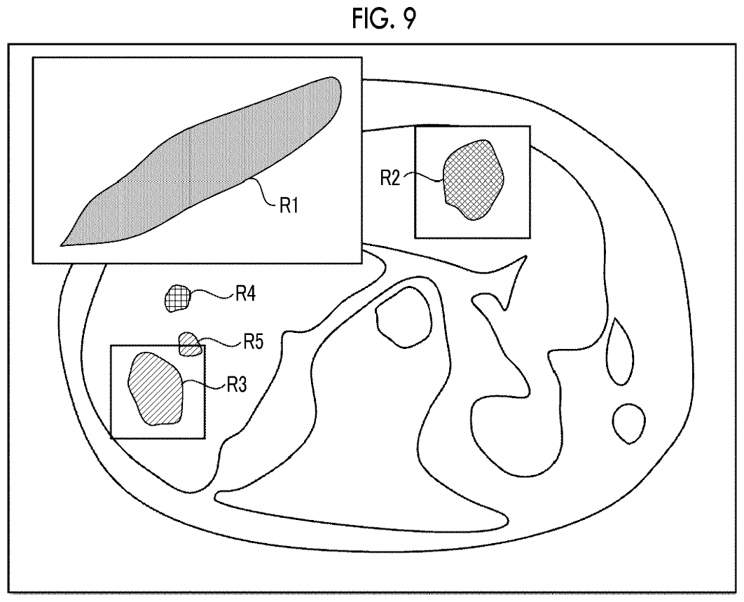

As shown in FIG. 9 as an example, the display control unit 48 may perform control to display the first status to the fourth status in an identifiable manner by the enlarged display or the reduced display. FIG. 9 shows an example in which the regions R1 and R2 whose status is the first status are enlarged and displayed.

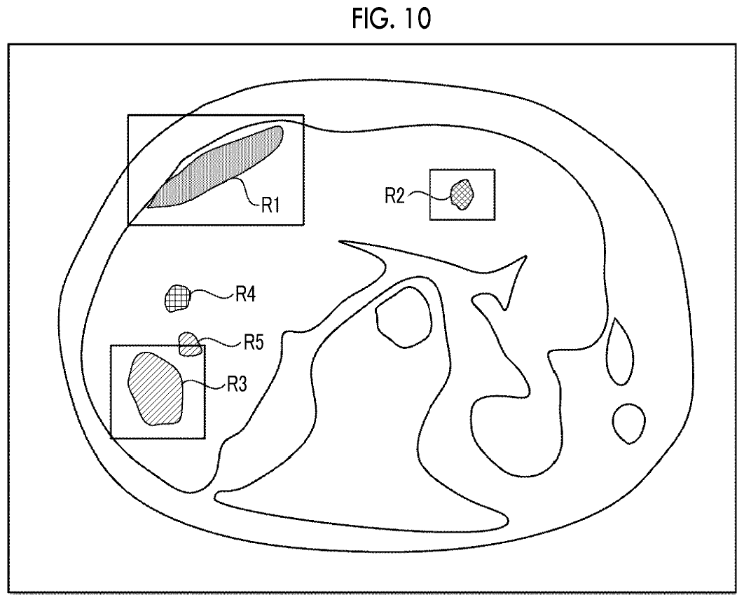

In addition, the display control unit 48 may perform control to display the first status and the second status among the first status to the fourth status to be different from the other statuses (that is, the third status and the fourth status). In this case, for example, the display control unit 48 performs control to highlight only the first status and the second status among the first status to the fourth status. As an example of the highlighting, as shown in FIG. 10, the region extracted by the extraction unit 42 is surrounded by a bounding box. FIG. 10 shows an example in a case where the statuses of regions R1 to R5 are the same as those in FIG. 6. In the example of FIG. 10, the regions R1 and R2 whose status is the first status and the regions R3 whose status is the second status are highlighted by being surrounded by bounding boxes. Note that the example of highlighting is not limited to the example shown in FIG. 10. For example, the display control unit 48 may perform control to highlight the region by filling the region with a preset color. In addition, for example, the display control unit 48 may perform control to highlight a region by drawing an outer edge of the region with a line of a preset color.

In addition, in a case where the user performs an end operation such as an operation of closing the status display screen in a state where a region whose status is the first status or the second status remains, the display control unit 48 may perform control to display a message to the effect that the creation work of the medical document is incomplete. Further, in this case, the user may be able to perform an operation of collectively setting the region whose status is the first status or the second status as the third status by one operation. Further, the display control unit 48 may perform control to display the image of the region portion as it is without performing additional display on the region whose status is the third status or the fourth status.

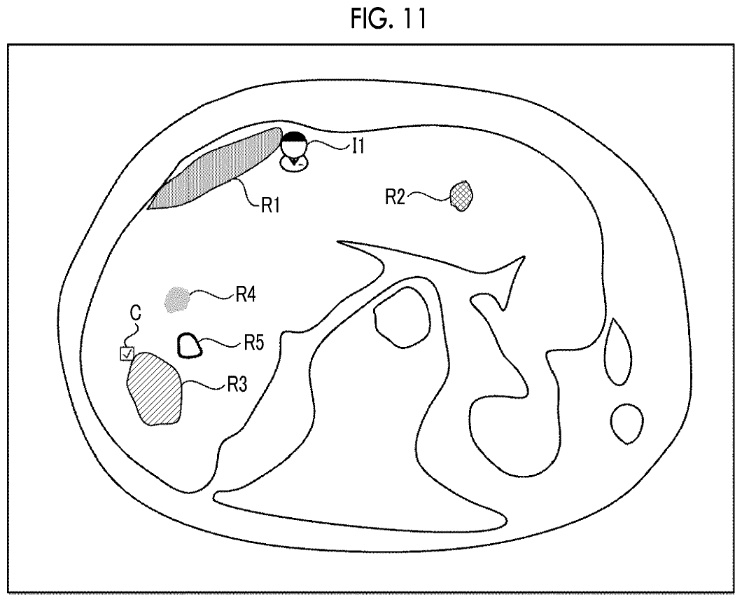

In addition, statuses other than the first status to the fourth status described above may be applied as the plurality of statuses related to the creation work of the medical document by the user. An example of the status in this case includes the status of being circulated to another doctor. FIG. 11 shows an example of a display screen of the status. In the example of FIG. 11, by displaying an icon Il representing a doctor in the vicinity of the region R1, the status of the region R1 is shown to be circulated to another doctor.

In addition, these plurality of statuses may be customizable for each hospital. For example, it may be possible to set which status is to be used for each hospital, or it may be possible to set a display mode for each status.

The reception unit 50 receives information indicating the region selected by the user from among the regions extracted by the extraction unit 42. In addition, the reception unit 50 receives an operation indicating which of the four statuses is set for the selected region. In addition, the reception unit 50 receives the comment on findings selected by the user from among the plurality of comments on findings displayed on the display 23 under the control of the display control unit 48. This received comment on findings is used to create a medical document.

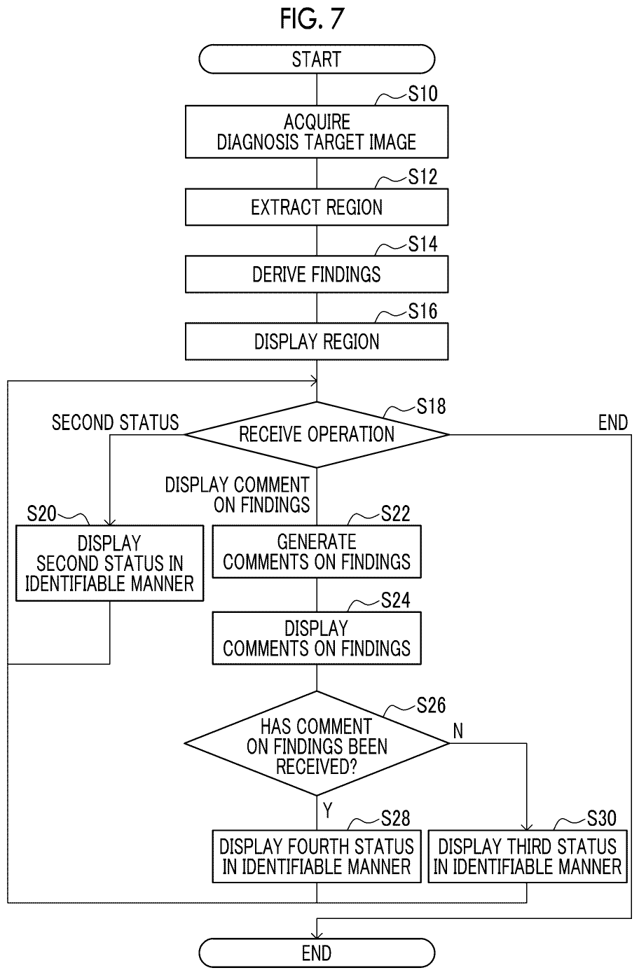

Next, with reference to FIG. 7, operations of the work support apparatus 10 according to the present embodiment will be described. The CPU 20 executes the work support program 30, whereby a work support process shown in FIG. 7 is executed. The work support process shown in FIG. 7 is executed, for example, in a case where an instruction to start execution is input by the user.

In Step S10 of FIG. 7, the acquisition unit 40 acquires the diagnosis target image from the image server 5 via the network OF 25. In Step S12, as described above, the extraction unit 42 extracts a region in the diagnosis target image acquired in Step S10 that is likely to be the target of the creation work of a medical document by a user such as a doctor. In Step S14, as described above, the analysis unit 44 analyzes each of the abnormal shadows extracted in Step S12, and derives findings of the abnormal shadows.

In Step S16, the display control unit 48 performs control to display information indicating the region extracted in Step S12 on the display 23. At the time of this control, as described above, the display control unit 48 performs control to display the status of each region to be identifiable as the first status. In Step S18, the reception unit 50 receives an operation by the user. In a case where the operation received by the reception unit 50 in Step S18 is an operation in which a region is designated by the user and the status of the designated region is set as the second status, the process proceeds to Step S20.

In Step S20, the display control unit 48 performs control to display the second status in an identifiable manner by adding a predetermined mark to the region designated by the user. In a case where the process of Step S20 ends, the process returns to Step S18.

In a case where the operation received by the reception unit 50 in Step S18 is an operation in which a region is designated by the user and an instruction for displaying the comment on findings is provided, the process proceeds to Step S22. In Step S22, as described above, the generation unit 46 generates a plurality of comments on findings based on the findings derived in Step S14 for the region designated by the user.

In Step S24, the display control unit 48 performs control to display the plurality of comments on findings generated in Step S22 on the display 23 for the region designated by the user. In Step S26, the reception unit 50 determines whether or not the comment on findings selected by the user from among the plurality of comments on findings displayed on the display 23 in Step S24 has been received. In a case where this determination is affirmative, the process proceeds to Step S28. In Step S28, the display control unit 48 performs control to display the fourth status in an identifiable manner by canceling the filling of the region designated by the user and drawing the outer edge of the region with lines of a predetermined color. In a case where the process of Step S28 ends, the process returns to Step S18.

In a case where the operation received by the reception unit 50 in Step S26 is an operation in which a region is designated by the user and the status of the designated region is set as the third status, the determination in Step S26 is a negative determination, and the process proceeds to Step S30. In Step S30, the display control unit 48 performs control to display the third status in an identifiable manner by graying out the region designated by the user. In a case where the process of Step S30 ends, the process returns to Step S18.

In a case where the operation received by the reception unit 50 in Step S18 is an operation to end the display of the screen, the work support process ends.

As described above, according to the present embodiment, it is possible to ascertain a user's work status for each region included in a medical image that is likely to be a work target by the user.

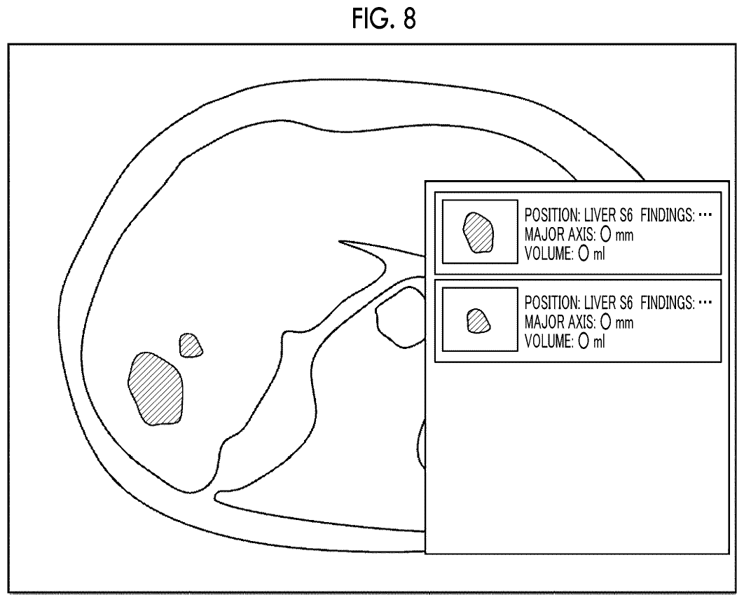

In the above embodiment, as shown in FIG. 8 as an example, the display control unit 48 may perform control to display a list of information regarding each of the plurality of regions extracted by the extraction unit 42 in a case where the user performs an operation to instruct display of the list. In this case, the display control unit 48 may perform control to display the list of the information regarding each of the plurality of regions extracted by the extraction unit 42 for each status.

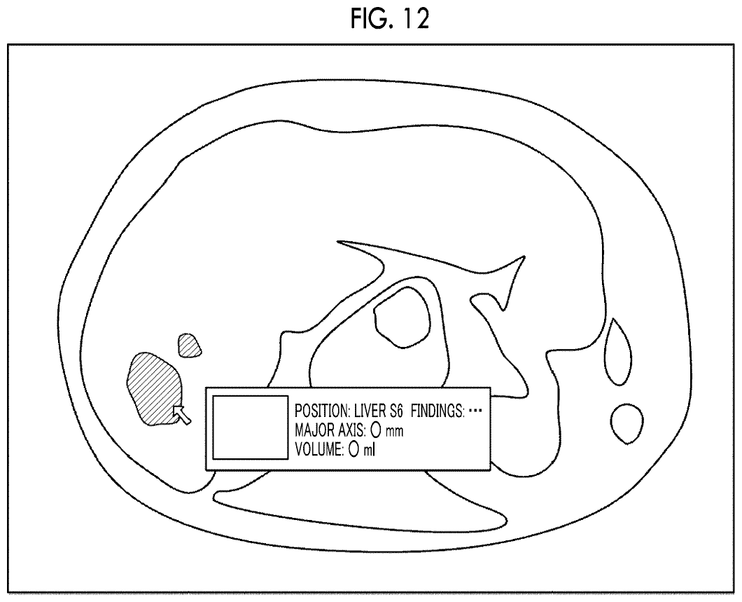

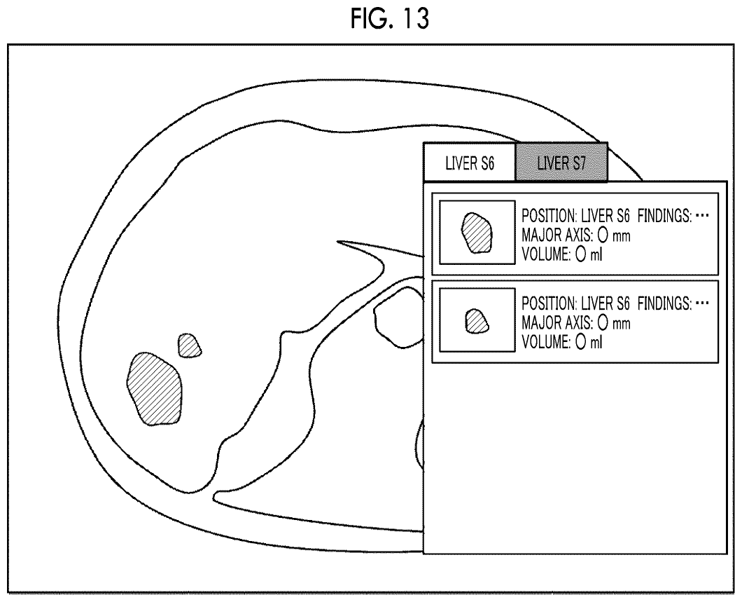

Further, the display control unit 48 may perform control to display information regarding each of the plurality of regions extracted by the extraction unit 42 in a display mode other than the list display. In this case, as shown in FIG. 12 as an example, the display control unit 48 may perform control to display information regarding the region extracted by the extraction unit 42 in a pop-up format. FIG. 12 shows an example in which information regarding a region designated by an arrow representing a mouse cursor is displayed in a pop-up format. Further, in this case, the display control unit 48 may perform control to display, in a pop-up format, information regarding a region having the same status as that of the designated region, not only in the designated region but also in the non-designated region. In addition, in this case, as shown in FIG. 13 as an example, the display control unit 48 may perform control to display information regarding each of the plurality of regions extracted by the extraction unit 42 in a tab format. FIG. 13 shows an example in which different tabs are provided for each region of the anatomical structure, and information regarding a region located within the anatomical structure region corresponding to the designated tab and extracted by the extraction unit 42 is displayed. In addition, FIG. 13 shows an example in which subsegments divided into S1 to S8 of the liver are applied as a region of the anatomical structure, and a tab of S6 of the liver is designated.

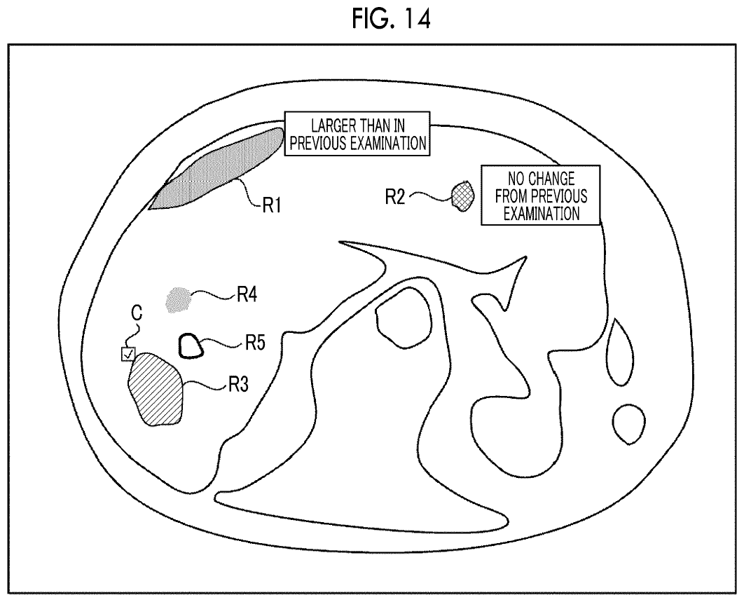

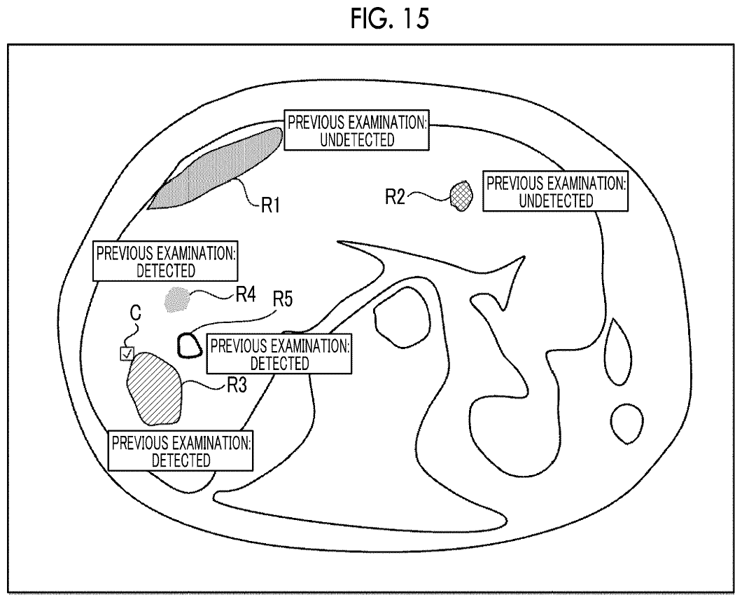

In addition, as shown in FIG. 14 as an example, the display control unit 48 may further perform control to display, for each of the plurality of regions extracted by the extraction unit 42, whether or not there is a change from the same region detected in the past examination in an identifiable manner. In this case, in order to ignore the error, the display control unit 48 may consider that there has been no change for a change equal to or less than a predetermined amount of change. FIG. 14 shows an example in which, in the regions R1 and R2 on the status display screen, the same region is extracted by the extraction unit 42 from the diagnosis target image captured in the previous examination. In addition, FIG. 14 shows an example in which the region R1 has a larger area than the diagnosis target image captured in the previous examination, and the region R2 has no change in area from the diagnosis target image captured in the previous examination. In addition, as shown in FIG. 15 as an example, the display control unit 48 may further perform control to display, for each of the plurality of regions extracted by the extraction unit 42, whether or not the same region has been detected in the past examination in an identifiable manner. FIG. 15 shows an example in which, in the regions R1 and R2, the same region is not extracted by the extraction unit 42 from the diagnosis target image captured in the previous examination. Further, FIG. 15 shows an example in which, in the regions R3 to R5, the same region is extracted by the extraction unit 42 from the diagnosis target image captured in the previous examination. In addition, the detected region referred to here means a region detected by an extraction process via a computer, such as an extraction process by the extraction unit 42, for example.

In addition, the display control unit 48 may further perform control to display, for each of the plurality of regions extracted by the extraction unit 42, whether or not there is a change from the same region reported in the past examination in an identifiable manner. In this case, in order to ignore the error, the display control unit 48 may consider that there has been no change for a change equal to or less than a predetermined amount of change. In addition, the display control unit 48 may further perform control to display, for each of the plurality of regions extracted by the extraction unit 42, whether or not the same region has been reported in the past examination in an identifiable manner. The reported region referred to here means, for example, a region not extracted by an extraction process via a computer, but discovered by a doctor and designated via the input device 24. That is, the reported region referred to here means a region that the doctor has previously reported to a medical document such as an interpretation report.

In addition, in the above embodiment, the case where the user selects one comment on findings from among a plurality of comments on findings has been described, but the present disclosure is not limited thereto. For example, the work support apparatus 10 may be configured to select one comment on findings from among a plurality of comments on findings based on the degree of certainty of the finding included in the comment on findings.

In the above embodiment, for example, as hardware structures of processing units that execute various kinds of processing, such as the acquisition unit 40, the extraction unit 42, the analysis unit 44, the generation unit 46, the display control unit 48, and the reception unit 50, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, there is a form in which a processor for realizing the function of the entire system including a plurality of processing units via one integrated circuit (IC) chip as typified by a system on chip (SoC) or the like is used. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the work support program 30 has been described as being stored (installed) in the storage unit 22 in advance; however, the present disclosure is not limited thereto. The work support program 30 may be provided in a form recorded in a recording medium such as a compact disc read only memory (CD-ROM), a digital versatile disc read only memory (DVD-ROM), and a universal serial bus (USB) memory. In addition, the work support program 30 may be configured to be downloaded from an external device via a network.

The disclosures of Japanese Patent Application No. 2021-068675 filed on Apr. 14, 2021 and Japanese Patent Application No. 2021-208526 filed on Dec. 22, 2021 are incorporated herein by reference in their entirety. In addition, all literatures, patent applications, and technical standards described herein are incorporated by reference to the same extent as if the individual literature, patent applications, and technical standards were specifically and individually stated to be incorporated by reference.

What is claimed is:

1. A work support apparatus comprising at least one processor,
wherein the processor is configured to:
acquire a medical image; and
perform control to display, in an identifiable manner, a status for a region that is likely to be a target of at least one work of interpretation work or creation work of a medical document by a user in the medical image among a plurality of statuses related to the work by the user.

2. The work support apparatus according to claim 1,
wherein the plurality of statuses include at least one of a status in which the work is required or a status in which the work is not required.

3. The work support apparatus according to claim 1,
wherein the plurality of statuses include two or more of a status in which the user has not confirmed the region, a status in which the user has designated the region as a target of the work and the work is incomplete, a status in which the user has designated that the region is excluded from the target of the work, and a status that the work for the region is completed.

4. The work support apparatus according to claim 3,
wherein the processor is configured to,
in a case where the status is the status in which the user has designated the region as the target of the work and the work is incomplete, perform control to display the status in an identifiable manner by adding a predetermined mark to the region.

5. The work support apparatus according to claim 3,
wherein the processor is configured to
perform control to display the status in which the user has not confirmed the region and the status in which the user has designated the region as the target of the work and the work is incomplete among the plurality of statuses to be different from other statuses.

6. The work support apparatus according to claim 1,
wherein the processor is configured to
further perform control to display information regarding each of a plurality of the regions.

7. The work support apparatus according to claim 6,
wherein the processor is configured
to perform control to display a list of the information regarding each of the plurality of regions.

8. The work support apparatus according to claim 7,
wherein the processor is configured to
perform control to display the list of the information regarding each of the plurality of regions for each of the statuses.

9. The work support apparatus according to claim 1,
wherein the region is a region including an abnormal shadow.

10. The work support apparatus according to claim 1,
wherein the region is extracted from the medical image by an extraction process via a computer.

11. The work support apparatus according to claim 1,
wherein the region is a region designated by the user.

12. The work support apparatus according to claim 1,
wherein the processor is configured to
further perform control to display a presence or absence of a change from the same region detected in a past examination for the region in an identifiable manner.

13. The work support apparatus according to claim 1,
wherein the processor is configured to
further perform control to display whether or not the same region has been detected in a past examination for the region in an identifiable manner.

14. The work support apparatus according to claim 1,
wherein the processor is configured to
further perform control to display a presence or absence of a change from the same region reported in a past examination for the region in an identifiable manner.

15. The work support apparatus according to claim 1,
wherein the processor is configured to
further perform control to display whether or not the same region has been reported in a past examination for the region in an identifiable manner.

16. A work support method executed by a processor provided in a work support apparatus, the method comprising:
acquiring a medical image; and
performing control to display, in an identifiable manner, a status for a region that is likely to be a target of at least one work of interpretation work or creation work of a medical document by a user in the medical image among a plurality of statuses related to the work by the user.

17. A non-transitory computer-readable storage medium storing a work support program for causing a processor provided in a work support apparatus to execute:
acquiring a medical image; and
performing control to display, in an identifiable manner, a status for a region that is likely to be a target of at least one work of interpretation work or creation work of a medical document by a user in the medical image among a plurality of statuses related to the work by the user.
